(19) United States
(12) Patent Application Publication
(10) Pub. No.: US 2013/0115286 A1
(43) Pub. Date: May 9, 2013

(54) ORAL ADMINISTRATION FORMS FOR CONTROLLED RELEASE OF RIFAMPICIN FOR THE TREATMENT OF BACTERIAL INFECTIONS AND INFLAMMATORY DISEASES OF THE GASTROINTESTINAL TRACT

(71) Applicants: Mario Brufani, Castelgandolfo (IT); Bianca Maria Lagrasta, Roma (IT); Rolando Marzella, Roma (IT); Ilaria Medici, Roma (IT); Silvio Silvestri, Roma (IT)

(72) Inventors: Mario Brufani, Castelgandolfo (IT); Bianca Maria Lagrasta, Roma (IT); Rolando Marzella, Roma (IT); Ilaria Medici, Roma (IT); Silvio Silvestri, Roma (IT)

(21) Appl. No.: 13/809,338

(22) PCT Filed: Jul. 13, 2011
(86) PCT No.: PCT/IB2011/053125

§ 371 Date: Jan. 9, 2013

(30) Foreign Application Priority Data
Jul. 13, 2010 (IT) MI2010A001283

(51) Int. Cl. A61K 31/496; A61K 31/438

(52) U.S. Cl. CPC A61K 31/496; A61K 31/438
USPC 424/463; 514/254.11; 514/278; 424/400; 424/475; 424/494; 424/497; 424/493; 424/495; 424/482; 424/480; 424/479; 427/2.21

(57) ABSTRACT

Oral administration forms are described for the controlled release of an antibiotic selected from the group consisting of rifampicin, rifabutin, rifapentine, rifalazil and mixtures thereof, for treating bacterial infections of the gastrointestinal tract, in particular travellers' diarrhoea, hepatic encephalopathy, ulcerative colitis, irritable bowel syndrome (IBS), Crohn's disease, and IBD (inflammatory bowel disease) in general. Moreover, said oral administration forms allow reduction of the amounts of antibiotic to be taken, with respect to the known administration forms and without reaching blood concentrations such as to select resistant strains of tuberculosis mycobacteria.

### FIELD OF THE INVENTION

The present invention relates to oral administration forms for controlled release of an antibiotic selected from the group comprising rifampicin, rifabutin, rifapentine, rifalazil and mixtures thereof, for treating bacterial infections and inflammatory diseases of the gastrointestinal tract, in particular travellers' diarrhoea, hepatic encephalopathy, ulcerative colitis, irritable bowel syndrome (IBS), Crohn's disease, and in general IBD (inflammatory bowel disease).

### BACKGROUND ART

Several works published in recent years have established that rifampicin, as well as being active against numerous bacterial strains, including Mycobacteria and bacteria responsible for intestinal infections including Clostridium difficile, activates the SXR nuclear receptor, inducing the transcription of several genes that code for phase 1 and 2 enzymes and antagonizing the activity of the nuclear receptor NF-kB, known to be responsible for the transcription of several genes that code for inflammatory proteins. In this way, activation of the SXR receptor by rifampicin is translated into a potent anti-inflammatory action on cells that express the SXR receptor, mainly intestinal and hepatic cells.

Inhibition of various bacterial strains, in particular Mycobacteria, that are present in the gastrointestinal tract requires high concentrations of rifampicin and related antibiotics; high concentrations of these antibiotics are also necessary for activating the SXR nuclear receptor.

Such concentrations of rifampicin are difficult to achieve in the intestine by administering the antibiotic by the oral route in the pharmaceutical forms currently on the market, which are quickly absorbed.

High concentrations of antibiotic would also be particularly useful for treating Crohn's disease, for which Mycobacterium avium subspecies paratuberculosis (MAP) is suggested as a possible causative agent. MAP is a rod-shaped asporogenous bacterium, which is slow-growing, and resistant to acid and alcohol, a characteristic that constitutes the basis for identifying the mycobacteria by Ziehl-Neelsen staining. The structure of the wall of Mycobacteria is formed from 4 layers, and the most superficial layer, consisting of glycolipids and lipoarabinomannan (LAM), has an important role in the pathogenesis of disease. Cell wall-deficient (CWD) strains, named spheroplasts, have been isolated in the tissues of patients with Crohn's disease. It appears that it is actually this form of modified wall that is the trigger for an abnormal immune response, which causes the disease. MAP has in fact been identified in samples of intestine from patients with Crohn's disease, an inflammatory disease with a multifactorial aetiology that affects young individuals (on average 15-25 years), thus suggesting a correlation between the presence of the microorganism and the disease (Chiodini R. J. (1989). Crohn's disease and the mycobacterioses: a review and comparison of two disease entities. Clinical Microbiology Reviews 2 90-117); the two conditions in fact have the same symptoms in common, i.e. general impairment, abdominal pain, chronic diarrhoea and weight loss caused by ileitis (Greenstein R. J. (2003). Is Crohn's disease caused by a mycobacterium? Comparisons with leprosy, tuberculosis, and Johne's disease. Lancet Infectious Diseases 3 507-514). Although there is uncertainty about the initial cause of the disease, it is known that environmental factors and genetic factors are also important. There is very active research into the genetic basis, and today the gene most implicated is NOD2, located on chromosome 16. A mutation of the gene causes a change in the capacity of the intestinal immune system to react appropriately to the presence of the bacteria that colonize the intestine. However, only 30% of patients with Crohn's disease have the NOD2 mutation, indicating that explanation of the part played by genetics is complex and that probably multiple genes are involved.

Moreover, MAP is also the aetiological agent responsible for paratuberculosis, an infectious, contagious and endemic disease that affects domestic and wild ruminants, as well as primates, and causes chronic-granulomatous enteritis. Investigated and described for the first time in 1895 by Johne and Frothingham, it is also known as Johne's disease. Paratuberculosis is one of the most important infectious diseases both for its wide occurrence and for the damage caused to cattle farming, where the type of stock-breeding, deficiencies in hygiene, boosts to production and the commercialization of animals have in recent years favoured the spread of infection and the increase in the incidence of the disease. It has been shown that, in populations living close to cattle farms, the frequency of Crohn's disease is higher than in the general population. In fact, transmission from animals to humans can occur via raw milk, heat-treated milk, or via meat, bearing in mind that, via the circulation, MAP can reach secondary organs such as lymph nodes, blood and, especially, muscle; water from natural water courses in areas where infected animals are pastured can also potentially be a vehicle.

A causal correlation between bacterial infection and Crohn's disease and IBS has not yet been found, but various clinical studies in which patients with these two pathologies were treated with rifampicin (or rifabutin) combined with another antitubercular agent (clarithromycin, clofazimine, ethambutol, isoniazid) show indisputable benefits.

MAP is a bacterial agent with little sensitivity to the action of antibiotics, and rifampicin and rifabutin are the antibiotics that are the most active against it, with MIC values between 1 and 2.5 μg/ml. Moreover, some strains of MAP, correlated with mutation of the rpoB gene (strains MAP18-MAP185 and UCF5-Rif 16 r), have shown resistance to rifampicin and to rifabutin; rifampicin has values of MIC (minimum inhibitory concentration) in the range 30-4 μg/ml and rifabutin in the range 10−1 μg/ml. (World J. of Gastr. 2008 ISSN 1007-9327, D. Beckler, et al.)

Since rifampicin, administered by the oral route at the maximum dose envisaged of 600 mg, gives blood levels not above 9 μg/ml, it can be assumed that the antibiotic administered by the oral route would not be able to eradicate intestinal infections with MAP as it does not reach the necessary inhibitory concentrations in the tissues of interest.

Rifampicin, represented as follows:

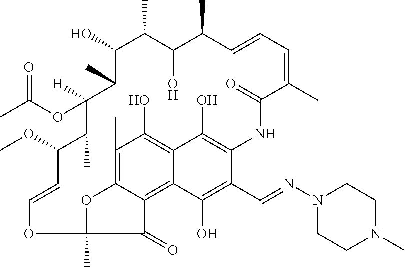

has a strong acid character, due to the presence of two hydroxyl groups in its molecule, which are located in the peri position on a napththohydroquinone ring, and has a pKa of 1.7. Therefore, when administered by the oral route, the antibiotic is absorbed mainly at acid pH, in the stomach and in the first portion of the duodenum. As confirmation of this, the antibiotic administered by the oral route to healthy volunteers at a dose of 600 mg reaches the maximum blood concentration (Cmax=9 μg/ml) after just 2 hours.

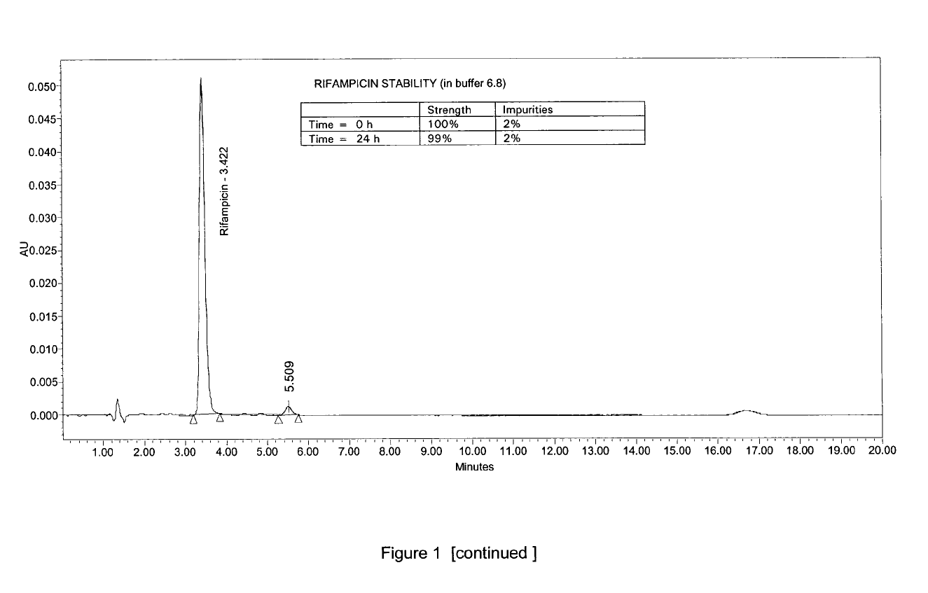

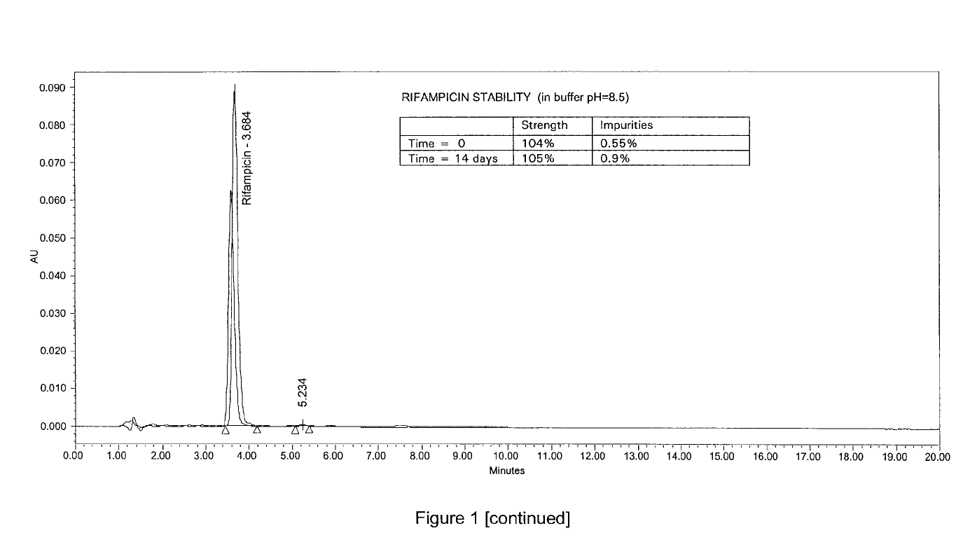

In the intestine, at neutral or basic pH, the antibiotic, being in the anionic form, should not be absorbed. It is in fact well known that in order to be absorbed, drugs such as rifampicin, which are not subject to active transport mediated by specific transporters, must pass through the phospholipid membranes of the parietal cells. In the case of substances with an acid character, such as rifampicin, only the undissociated form of the antibiotic is able to pass quickly through the phospholipid membranes of the parietal cells and hence be absorbed. The percentage of an acid present in undissociated form, which can be calculated from the well-known Henderson-Hasselbalch equation, depends on the difference between the pKa value of the acid and the pH of the environment. It follows from the aforementioned equation that for rifampicin at pH 5.5 the ratio between the percentages of it in ionic form and undissociated form is about 10.000/1. Therefore in alkaline and neutral environments, as the antibiotic is completely in ionic form it has little chance of being absorbed. Regarding stability, rifampicin is sufficiently stable in neutral and weakly alkaline environments, and less stable in an acid environment, as shown in FIG. 1.

Pharmacokinetic studies carried out with variable doses of rifampicin between 100 and 600 mg on patients and healthy volunteers, also reported recently in the literature (Clinical Pharmacokinetics of Rifampicin, Acocella, page 114) have shown that in 12 hours approx. 20% of the dose of rifampicin taken by the oral route is eliminated in the urine. In addition, a study published in “The International Journal of Turberculosis and Lung Disease” (Vol. 5, No. 8, August 2001, pages 691-695) demonstrated, on statistical bases, by a randomized clinical study, that there is excellent correlation between the data on bioavailability of rifampicin, based on measurements of blood concentrations and concentrations in urinary excretion. Consequently, the data on urinary excretion can be used as an alternative to the haematic data for evaluating the bioavailability of various formulations of rifampicin.

The object of the present invention is therefore to make high concentrations of rifampicin, and of other related antibiotics, such as rifabutin, rifapentine, rifalazil, available locally in sections of the intestine affected by chronic bacterial infections, caused in particular by MAP, with particular reference to Crohn's disease, or by other bacteria, such as Clostridium difficile, for ulcerative and pseudomembranous colitis. Clostridium difficile is a Gram-positive anaerobic bacterium, so it generally survives well in our intestine, which is low in oxygen, especially in the large intestine. In fact, many people have Clostridium difficile in a latent form; when something upsets the normal intestinal balance, the bacterium can proliferate uncontrollably, and begin to release toxins that attack the intestinal mucosa: this leads to symptoms of Clostridium difficile infection with consequent production of toxins that will interact with the enterocytes, glycosylating numerous proteins and triggering apoptotic mechanisms. The release of cytokines accompanying cell death calls a large number of immune defence cells to the site, which, maintaining the state of inflammation, will damage the cells further. The accumulation of cellular debris, mucus etc. on the intestinal mucosa produces the pseudomembrane that will gradually expand over the intestinal mucosa, with consequent manifestation of pseudomembranous colitis.

### SUMMARY OF THE INVENTION

The object above has been achieved by an oral administration form, as in claim 1, comprising:

a solid mixture of an antibiotic selected from the group consisting of rifampicin, rifabutin, rifapentine, rifalazil and mixtures thereof, and pharmaceutically acceptable excipients, and

a coating in an amount of 6 to 20 mg/cm², said coating comprising an enteric polymer selected from the group consisting of cellulose acetate phthalate, cellulose acetate succinate, hydroxypropylmethylcellulose phthalate, hydroxypropylmethylcellulose acetate succinate, polyvinyl acetate phthalate, hydroxyethylcellulose phthalate, cellulose acetate tetrahydrophthalate, methacrylate-methacrylic acid copolymers, methylmethacrylate-methacrylic acid copolymers, sodium alginate, stearic acid and mixtures thereof, and pharmaceutically acceptable excipients.

In another aspect, the present invention relates to a unit dose of said oral administration form comprising antibiotic from 100 to 600 mg.

In a further aspect, the present invention relates to a process for preparing the oral administration form or the unit dose thereof, comprising the steps of:
a) providing a solid mixture of antibiotic and excipients; and
b) applying the coating up to an amount of 6 to 20 mg/cm² on said solid mixture.

In a even further aspect, the present invention relates to the use of said oral administration form or its unit dose for treating chronic bacterial infections, in particular travellers' diarrhoea, hepatic encephalopathy, ulcerative colitis, irritable bowel syndrome (IBS), Crohn's disease, and in general IBD (inflammatory bowel disease).

### BRIEF DESCRIPTION OF THE DRAWING

The characteristics and advantages of the present invention will be clear from the detailed description given below, the examples given for illustrative and non-limiting purposes, and the appended FIG. 1, which shows the stability tests on rifampicin in acid, neutral and basic environments.

### DETAILED DESCRIPTION OF THE INVENTION

The invention therefore relates to an oral administration form comprising:
a solid mixture of an antibiotic selected from the group comprising rifampicin, rifabutin, rifapentine, rifalazil and mixtures thereof, and pharmaceutically acceptable excipients, and
a coating in an amount of from 6 to 20 mg/cm², said coating comprising an enteric polymer selected from the group comprising cellulose acetate phthalate, cellulose acetate succinate, hydroxypropylmethylcellulose phthalate, hydroxypropylmethylcellulose acetate succinate, polyvinyl acetate phthalate, hydroxyethylcellulose phthalate, cellulose acetate tetrahydrophthalate, methacrylate-methacrylic acid copolymers, methylmethacrylate-methacrylic acid copolymers, sodium alginate, stearic acid and mixtures thereof, and pharmaceutically acceptable excipients.

It has in fact been observed that the coating of the solid mixture comprising the polymer as above advantageously allows intestinal levels of antibiotic to be reached that are sufficient to eradicate bacterial infections, caused primarily by MAP, while the blood concentrations remain conveniently extremely low. In fact, the oral administration form of the present invention releases the antibiotic locally in the intestine, as it is resistant to the acid environment of the stomach and disintegrates at pH between 3 and 8. In particular, this oral administration form allows high concentrations of antibiotic to be reached in the intestine, bearing in mind that in particular rifampicin has a solubility in the intestine determined by the pH values of the order of 1-6 mg/ml, and at the same time blood concentrations that are advantageously insufficient to select resistant strains of tuberculosis mycobacteria, which are sensitive to higher concentrations of the antibiotic. Moreover, taking into account the solubility of the drug and the average volumes of the intestinal fluids, the doses of said drug sufficient to saturate the intestinal fluids may be greatly reduced, as will be seen more clearly from the examples given below.

Thus, this oral administration form has surprisingly proved to be suitable for controlled release of antibiotic for treating bacterial infections of the gastrointestinal tract, in particular travellers' diarrhoea, hepatic encephalopathy, ulcerative colitis, irritable bowel syndrome (IBS), Crohn's disease, and in general IBD (inflammatory bowel disease), where “controlled release” means that this administration form is able to release the antibiotic in predefined regions of the intestine as a function of the pH present therein.

According to a preferred embodiment, said oral administration form consists of said solid mixture and said coating.

According to a more preferred embodiment, said oral administration form consists of said solid mixture and said coating, wherein the coating consists of said enteric polymer and pharmaceutically acceptable excipients.

Preferably, said enteric polymer is in an amount of at least 30 wt. % based on the dry weight of the coating.

According to a first preferred embodiment, said enteric polymer is in an amount of at least 95 wt. % based on the dry weight of the coating, and said coating is in an amount of 8 to 18.6 mg/cm². It was in fact observed that these amounts resist unchanged at strongly acid pH, while advantageously they release the antibiotic at pH between 4-6 in desirably and conveniently faster times.

According to another preferred embodiment, said enteric polymer is in an amount of 35 to 70 wt. % based on the dry weight of the coating. This embodiment envisages that the coating of the solid mixture is apportioned in a first layer consisting of the excipients only and in a second layer consisting of the enteric polymer and the excipients. In this case, said enteric polymer is preferably in an amount of 50 to 80 wt. % based on the dry weight of the second layer, more to preferably 60 to 70 wt. % based on the dry weight of the second layer. Alternatively, said coating is apportioned in a first layer consisting of a first enteric polymer and pharmaceutically acceptable excipients and in a second layer consisting of a second enteric polymer and pharmaceutically acceptable excipients, said first and second enteric polymers being identical or different. Preferably, the amount of enteric polymer in the second layer is greater than the amount of enteric polymer in the first layer; more preferably, the amount of enteric polymer in the second layer is at least 5 times the amount of enteric polymer in the first layer.

Preferably, said oral administration form is capsule, tablet, mini-tablet, micro-tablet, granule, microgranule, pellet, multiparticulate, micronized particulate. More preferably, said oral administration form is capsule.

Suitable pharmacologically acceptable excipients for the solid mixture are diluents, disintegrants, glidants, binders, lubricants, stabilizers, adsorbents, release retardants and preservatives. In particular, the following are used for the purposes of the present invention: natural starch, partially hydrolysed starch, modified starch, triethyl citrate, glycerol, potassium sorbate, lactose, glucose, sucrose, mannitol, sorbitol, cellulose and derivatives thereof, microcrystalline cellulose and derivatives thereof, calcium phosphate, calcium carbonate, calcium sulphate, polyvinylpyrrolidone, gelatin, gum tragacanth, gum arabic, polyethylene glycol, alginates, talc, magnesium stearate, silica, colloidal silica, precipitated silica, magnesium silicates, aluminium silicates, sodium lauryl sulphate, magnesium lauryl sulphate, methacrylate copolymers and mixtures thereof.

In another aspect, the present invention relates to a unit dose of said oral administration form comprising 100 to 600 mg of antibiotic.

Preferably, said unit dose comprises 150 to 300 mg of antibiotic. In fact, as already mentioned, the oral administration form of the present invention allows the amount of antibiotic required for treating bacterial infections in the intestine to be reduced appreciably, at the same time maintaining blood concentrations that are advantageously insufficient to select resistant strains of tuberculosis mycobacteria. According to a preferred embodiment of the invention, said unit dose is a capsule. In a further aspect, the present invention relates to a process for preparing the oral administration form or the unit dose as above, comprising the steps of:
a) providing a solid mixture of antibiotic and excipients; and
b) applying the coating up to an amount of 6 to 20 mg/cm² on said solid mixture.

According to a preferred embodiment, said step b) is performed by film coating in a coating pan.

Preferably, said step b) is carried out in two sub-steps:
b-i) applying a first coating up to an amount of 4 to 8 mg/cm² on said solid mixture; and
b-ii) applying a second coating up to an amount of 2 to 12 mg/cm².

In fact, it has been observed that a first partial apportioning of the coating allows the external surface of the solid mixture to be made uniform, while the second partial apportioning of the coating makes it possible to reach a total amount of 6 to 20 mg/cm², according to the present invention.

More preferably, said step b) is carried out in two sub-phases:
b-i) applying a first coating up to an amount of 5 to 7 mg/cm² on said solid mixture; and
b-ii) applying a second coating up to an amount of 2 to 12 mg/cm².

According to a preferred embodiment of the invention, said first coating consists of pharmaceutically acceptable excipients, the enteric polymer being entirely in the second coating. In this case, preferably, said enteric polymer is in an amount of 50 to 80 wt. % based on the dry weight of the second coating, more preferably 60 to 70 wt. % based on the dry weight of the second coating.

Alternatively, said first coating comprises an enteric polymer identical to or different from that of said second coating. Preferably, the amount of enteric polymer in the second coating is higher than the amount of enteric polymer in the first coating; more preferably, the amount of enteric polymer in the second coating is at least 5 times the amount of enteric polymer in the first coating.

In a further aspect, the present invention relates to the use of said oral administration form for controlled release of antibiotic in the treatment of chronic bacterial infections, in particular travellers' diarrhoea, hepatic encephalopathy, ulcerative colitis, irritable bowel syndrome (IBS), Crohn's disease, and in general IBD (inflammatory bowel disease). Said oral administration form, besides ensuring a high concentration of antibiotic in the intestine, advantageously does not select antibiotic-resistant strains of tuberculosis mycobacteria in the respiratory system, since the blood concentrations of antibiotic, zero or at least very low, are insufficient to select resistant strains.

Preferably, said use comprises the administration of 100 to 600 mg of rifampicin per day, more preferably 150 to 300 mg of rifampicin per day.

Working examples the present invention are given below for non-limiting illustrating purposes.

### EXAMPLES

### Example 1

### Preparation of the Oral Administration Forms According to the Present Invention

The solid mixtures of powders shown below in Table 1 were prepared.

TABLE 1

| | Rifampicin | Excipients |
|---|---|---|
| a | 100 mg | 25 mg of maize starch; |
| | | 25 mg of microcrystalline cellulose; |
| | | 10 mg of talc |
| b | 200 mg | 25 mg of maize starch; |
| | | 25 mg of microcrystalline cellulose; |
| | | 10 mg of talc |
| c | 300 mg | 25 mg of maize starch; |
| | | 25 mg of microcrystalline cellulose; |
| | | 10 mg of talc |
| d | 400 mg | 30 mg of maize starch; |
| | | 30 mg of microcrystalline cellulose; |
| | | 10 mg of talc |
| e | 500 mg | 30 mg of maize starch; |
| | | 35 mg of microcrystalline cellulose; |
| | | 15 mg of talc |
| f | 600 mg | 35 mg of maize starch; |
| | | 35 mg of microcrystalline cellulose; |
| | | 20 mg of talc |

All the solid mixtures given above were obtained by mixing and homogenizing the compounds, contained in hard gelatin capsules and then film-coated, comprising >99 wt. % of cellulose acetate phthalate, in increasing amounts, as follows:

3.0 mg/cm²

5.3 mg/cm²

8.8 mg/cm²

12.3 mg/cm²

15.4 mg/cm²

18.6 mg/cm².

### Example 2

### Disintegration Test According to the European Pharmacopoeia (EP) of the Oral is Administration Forms Prepared in Example 1

The disintegration test according to the EP for enteric capsules was carried out on the capsules of Example 1, applying the single variant of the disintegration medium following 0.1N hydrochloric acid.

In fact, the EP currently requires that the enteric capsules are to be placed in the prescribed apparatus using 0.1N HCl as disintegrating liquid, operating the apparatus for 2 hours. At the end of this period, the liquid is replaced with buffer at pH 6.8 and the apparatus is restarted.

For the present test, a buffer was instead used with a pH more suitable for the purposes of the present invention, i.e. pH=5.

The capsules obtained in Example 1 were then put in the apparatus for the disintegration test for 2 hours with 0.1N hydrochloric acid. After two hours, those that had not shown breakdown of the film coating were put in the buffer solution at pH=5 and the disintegration times in this second medium were recorded.

The results obtained from these tests are schematized in the following Table 2.

TABLE 2

| Amount of | | |
|---|---|---|
| coating | Disintegration in | Disintegration in |
| (mg/cm²) | 0.1N HCl | buffer sol. at pH 5 |
| 3.0 | Some capsules open after | / |
| | about 60 minutes | |
| 5.3 | Some capsules open after | / |
| | about 90 minutes | |
| 8.8 | The capsules are still closed | / |
| | at the end of two hours, but | |
| | some are deformed | |
| 12.3 | The capsules are unchanged | The capsules open |
| | at the end of two hours | after about 15 minutes |
| 15.4 | The capsules are unchanged | The capsules open |
| | at the end of two hours | after about 40 minutes |
| 18.6 | The capsules are unchanged | The capsules open |
| | at the end of two hours | after more than 60 |
| | | minutes |

By analysing the results in Table 2, it can be understood that the oral administration forms that best satisfied the purposes of the present invention at pH 5 were those comprising about 12 mg/cm² of enteric polymer.

### Example 3

### Preparation of the Oral Administration Forms According to the Present Invention

The following solid mixtures of powders were prepared, as shown in Table 3.

TABLE 3

| | Drug | Excipients |
|---|---|---|
| g | 120 mg of Rifabutin | 50 mg of lactose monohydrate; |
| | | 10 mg of magnesium stearate; |
| | | 12 mg of anhydrous colloidal silica |
| h | 150 mg of Rifampicin | 43 mg of lactose monohydrate; |
| | | 5 mg of magnesium stearate; |
| | | 2 mg of anhydrous colloidal silica |
| i | 180 mg of Rifapentine | 45 mg of lactose monohydrate; |
| | | 8 mg of magnesium stearate; |
| | | 6 mg of anhydrous colloidal silica |
| l | 200 mg of Rifalazil | 80 mg of lactose monohydrate; |
| | | 35 mg of magnesium stearate; |
| | | 25 mg of anhydrous colloidal silica |
| m | 250 mg of Rifabutin | 60 mg of lactose monohydrate; |
| | | 20 mg of magnesium stearate; |
| | | 15 mg of anhydrous colloidal silica |
| n | 300 mg of Rifampicin | 75 mg of lactose monohydrate; |
| | | 15 mg of magnesium stearate; |
| | | 30 mg of anhydrous colloidal silica |

All the solid mixtures given above, each corresponding to a unit dose, were obtained by mixing and homogenizing the compounds, and were enclosed in hard gelatin capsules.

For each composition g-n shown above, 270 capsules were prepared, and were then divided into 9 groups.

All the capsules were then submitted to film coating in a coating pan, as follows:

b-i) for all the capsules, a first film coating was carried out with the excipients: modified starch, talc, glycerol, potassium sorbate, and propane-1,2-diol alginate, up to an amount of coating of 6 mg/cm²;
b-ii) then a second film coating was carried out with methacrylic acid-ethyl acrylate copolymer (1:1) (commercially available as EUDRAGIT® L 30 D-55), and excipients: talc, triethyl citrate (TEC), potassium sorbate, and propane-1,2-diol alginate (commercially available as W.A.S.—L Pigment Suspension Ready to Use); the copolymer was present at 62.2 wt. % based on the weight of the second film coating.

This second film coating had different amounts for each of the 9 capsule groups of each solid mixture previously prepared, as shown in Table 4.

TABLE 4

| Groups for each | Amount for first | Amount for second | Total amount |
|---|---|---|---|
| mixture g-n | film coating | film coating | of coating |
| 1 | 6 mg/cm² | 2 mg/cm² | 8 mg/cm² |
| 2 | 6 mg/cm² | 3 mg/cm² | 9 mg/cm² |
| 3 | 6 mg/cm² | 4 mg/cm² | 10 mg/cm² |
| 4 | 6 mg/cm² | 5 mg/cm² | 11 mg/cm² |
| 5 | 6 mg/cm² | 6 mg/cm² | 12 mg/cm² |
| 6 | 6 mg/cm² | 7 mg/cm² | 13 mg/cm² |
| 7 | 6 mg/cm² | 8 mg/cm² | 14 mg/cm² |
| 8 | 6 mg/cm² | 9 mg/cm² | 15 mg/cm² |
| 9 | 6 mg/cm² | 10 mg/cm² | 16 mg/cm² |

### Example 4

### Disintegration Test According to the European Pharmacopoeia (EP) of the Oral Administration Forms Prepared in Example 3-h

The disintegration test according to the EP for enteric capsules was carried out on the 9 groups of capsules coated according to Example 3-h.

For the groups of capsules from 1 to 4 that did not satisfy the disintegration test according to the EP, a second disintegration test was carried out, wherein buffers at various pH values were used for verifying the behaviour of the capsules of the invention with different amounts of coating with variation of pH.

All the capsules were put in the apparatus for the disintegration test for 1 hour with 0.1N hydrochloric acid and for 30 minutes each at pH from 3 to 6.8. The results obtained from these tests are presented in Table 5 below.

TABLE 5
Second disintegration test

| Total | | Buffer | Buffer | Buffer | Buffer | Buffer |
|---|---|---|---|---|---|---|
| amount of | 0.1N HCl | solution | solution | solution | solution | solution |
| coating | (60 min) | pH 3 | pH 4 | pH 5 | pH 6 | pH 6.8 |
| 8 mg/cm² | 6 cps open | | | | | |
| | in 30 min | | | | | |
| 9 mg/cm² | 4 cps open | | | | | |
| | in 40 min | | | | | |
| | 2 cps open | | | | | |
| | in 60 min | | | | | |
| 10 mg/cm² | 1 cps open | 1 cps open | 2 cps open | | | |
| | in 40 min | in 20 min | in 10 min | | | |
| | 5 cps closed | 4 cps closed | 2 cps open | | | |
| | | | in 30 min | | | |
| 11 mg/cm² | 6 cps closed | 6 cps closed | 1 cps open | 5 cps closed | 6 cps open | |
| | | | in 30 min | | in 15 min | |
| | | | 5 cps closed | | | |
| 12 mg/cm² | 6 cps closed | 6 cps closed | 6 cps closed | 6 cps closed | 6 cps open | |
| | | | | | in 20 min | |
| 13 mg/cm² | 6 cps closed | 6 cps closed | 6 cps closed | 6 cps closed | 2 cps open | |
| | | | | | in 15 min | |
| | | | | | 4 cps open | |
| | | | | | in 30 min | |
| 14 mg/cm² | 6 cps closed | 6 cps closed | 6 cps closed | 6 cps closed | 1 cps open | 5 cps open |
| | | | | | in 20 min | in 18 min |
| | | | | | 5 cps closed | |

The film-coated capsules were submitted to the disintegration test by using modified times and media in order to simulate as far as possible the gastrointestinal course as regards the pH values of the various media used, by setting for each of them, times that might provide adequate indications with regard to the position in which the capsule tended to open in the course of passage through the intestine. The results showed that there is a correlation between the thickness of film coating and time of opening of the capsules, allowing modulation of release of the contents of the capsules in the various sections of the digestive system.

Then disintegration tests according to the EP were repeated, as shown in the following Table 6.

TABLE 6
Disintegration test according to EP

| Total | | Buffer |
|---|---|---|
| amount of | 0.1N HCl | solution |
| coating | (2 hours) | pH 6.8 |
| 12 mg/cm² | 6 cps closed | 6 cps open |
| | | in 6 min |
| 13 mg/cm² | 6 cps closed | 6 cps open |
| | | in 10 min |
| 14 mg/cm² | 6 cps closed | 6 cps open |
| | | in 14 min |
| 15 mg/cm² | 6 cps closed | 6 cps open |
| | | in 15 min |
| 16 mg/cm² | 6 cps closed | 6 cps open |
| | | in 20 min |

As can be seen from Table 6 above, on some thicknesses of film coating that on first examination gave disintegration times compatible with true gastro-resistance, the test was carried out according to the EP specification, keeping the capsules in 0.1N HCl for two hours and then putting them in buffer at pH 6.8 and recording the disintegration times of the capsules. It followed from these results that with total thicknesses of film coating of 12 mg/cm² it was already possible to obtain sufficient characteristics of gastro-resistance and that moreover, independently of the increase in thickness (up to 16 mg/cm²), release of the active substance occurred in the last section of the intestine within the times stipulated by the EP.

By analysing the results in Table 5 and Table 6, it can be understood that the oral administration forms of the invention could be coated with amounts of coating, within the overall range 6-20 mg/cm², as a function of the pH at which it is desirable for the antibiotic to be released, each pH being identifiable in defined regions of the intestine.

### Example 5

### Test Comparing the Oral Administration Forms Prepared in Example 1 and Known Tablets of Rifampicin

A group of healthy adult volunteers was selected, and they were administered two commercially available 300 mg tablets of rifampicin and, after a wash-out period of 1 week, were administered 2 capsules as in Example 1-c, i.e. each containing 300 mg of rifampicin, with a film coating in an amount of 12.3 mg/cm².

Measurements of elimination in the urine were performed, which can be correlated, as already mentioned, with the blood levels, for evaluating the level of bioavailability of rifampicin. The volunteers' urine was collected at two-hour intervals (as described below) and examined for rifampicin content. The 24-hour urine of each patient was then collected, concentrated and also examined for rifampicin content. None of the individual urine samples taken from the volunteers to whom the two tablets in example 1-c had been administered (amount of film coating 12.3 mg/cm²) was found to have an amount of rifampicin detectable with the analytical methods that we used. Total amounts of rifampicin not exceeding 6 μg were found in the total urine (collected for 24 hours after administration).

It was deduced from the measurements of elimination in the urine that, at urinary recovery≦6 μg, the total blood content of rifampicin should be ≦30 μg. In fact, it is known from the literature that the amount of rifampicin present in the plasma is 5 times higher than that eliminated by the renal route. Taking into account that an is adult individual has about 6 litres of blood, the plasma concentrations of rifampicin should therefore be ≦0.005 μg/ml.

The experiment was conducted by collecting the urine for each volunteer at intervals of 2 hours starting from the time of administration up to the fourteenth hour and a last check sample 24 hours after administration (see Table 7 below). In addition, stool samples were obtained on the next day after administration. The levels of rifampicin present in the samples were determined by HPLC, after acidification of said samples, extraction with chloroform and/or ethyl acetate and successive washings until the organic phase is neutralized, drying of the organic phase over sodium sulphate and then evaporation to dryness (T<40° C. and reduced P mmHg). The residue was taken up in a suitable solvent for HPLC. HPLC analysis was carried out using a Waters liquid chromatograph equipped with UV detector at variable wavelength and set at 254 nm, by the RP technique with Phenomenex Luna C18 5μ column (100*4.6 mm) and isocratic elution with a mobile phase composed of a 70/30 mixture of phosphate buffer at pH 3.5 and acetonitrile. The working flow was 1.5 ml/min.

### Results

In the case of urine, the current limit of quantification of the antibiotic was of the order of 0.1 μg/ml. Measurable levels of rifampicin were not detected in any fractions of urine collected from subjects treated with the capsules of Example 1-c (amount of film coating 12.3 mg/cm²), i.e. no sample exceeded the threshold of detectability of the antibiotic. The results of this test are presented in Table 7.

TABLE 7
Results for rifampicin recovery by the renal route

| | Treatment with 2 | Treatment with 2 known |
|---|---|---|
| | capsules of Example 1-c | 300-mg tablets of |
| Sample | (μg/ml) | Rifampicin (μg/ml) |
| Urine test after: 0 h | <LOQ | <LOQ |
| Urine test after: 2 h | <LOQ | 2.2 |
| Urine test after: 4 h | <LOQ | 29.3 |
| Urine test after: 6 h | <LOQ | 30.1 |
| Urine test after: 8 h | <LOQ | 13.1 |
| Urine test after: 14 h | <LOQ | 11.1 |
| Urine test after: 24 h | <LOQ | 0.8 |

LOQ (limit of quantification) = 0.1 μg/ml

For each volunteer, treated with the capsules in Example 1-c, the urine samples were combined (with volumes of the order of 600-800 ml), acidified, neutralized and extracted several times with chloroform and ethyl acetate. The amounts of rifampicin recovered in the total urine of each volunteer were between 2 and 6 μg. This result confirmed the limited absorption of rifampicin administered by the oral route of the formulation of Example 1-c. An extremely low content of rifampicin in is the total urine also confirmed that the amount of antibiotic absorbed and present in the plasma was similarly very low.

Conversely, detectable amounts of unchanged rifampicin were measured in the volunteers' faeces. The presence of rifampicin in the volunteers' faeces was confirmed in small stool samples.

The advantages achieved with the oral administration forms of the present invention can be seen from the detailed description and from the examples given above. In particular, these oral administration forms were found, surprisingly and advantageously, to be able to make the antibiotics available locally in the sections of the intestine affected by chronic bacterial infections, caused mainly by MAP, with particular reference to Crohn's disease and by Clostridium difficile for colitis, for which they represent the most effective antibiotics. Moreover, these oral administration forms allow a reduction in the amounts of antibiotic to be taken, relative to known administration forms, and without reaching blood concentrations such that resistant strains of tuberculosis mycobacteria are selected.

1. Oral administration form comprising:
a solid mixture of an antibiotic selected from the group consisting of rifampicin, rifabutin, and mixtures thereof, and pharmaceutically acceptable excipients, and
a coating in an amount of 6 to 20 mg/cm², said coating comprising an enteric polymer selected from the group consisting of cellulose acetate phthalate, cellulose acetate succinate, hydroxypropylmethylcellulose phthalate, hydroxypropylmethylcellulose acetate succinate, polyvinyl acetate phthalate, hydroxyethylcellulose phthalate, cellulose acetate tetrahydrophthalate, methacrylate-methacrylic acid copolymers, methylmethacrylate-methacrylic acid copolymers, sodium alginate, stearic acid and mixtures thereof, and pharmaceutically acceptable excipients.

2. The oral administration form of claim 1, wherein said enteric polymer is in an amount of at least 30 wt. % based on the dry weight of the coating.

3. The oral administration form of claim 2, wherein said enteric polymer is in an amount of at least 95 wt. % based on the dry weight of the coating, and said coating is in an amount of 8 to 18.6 mg/cm².

4. The oral administration form of claim 2, wherein said enteric polymer is in an amount of 35 to 70 wt. % based on the dry weight of the coating, said coating being apportioned in a first layer consisting only of the pharmaceutically acceptable excipients and in a second layer consisting of enteric polymer and pharmaceutically acceptable excipients.

5. The oral administration form of claim 4, wherein said enteric polymer is in an amount of 50 to 80 wt. % based on the dry weight of said second layer.

6. The oral administration form of claim 5, wherein said enteric polymer is in an amount of 60 to 70 wt. % based on the dry weight of said second layer.

7. The oral administration form of claim 2, wherein said enteric polymer is in an amount of 35 to 70 wt. % based on the dry weight of the coating, said coating being apportioned in a first layer consisting of a first enteric polymer and pharmaceutically acceptable excipients and in a second layer consisting of a second enteric polymer and pharmaceutically acceptable excipients, said first and second enteric polymers being identical or different.

8. The oral administration form of claim 1, wherein said oral administration form is capsule, tablet, mini-tablet, micro-tablet, granule, microgranule, pellet, multiparticulate, or micronized particulate.

9. The oral administration form of claim 8, wherein said oral administration form is capsule.

10. The oral administration form of claim 1, wherein said pharmacologically acceptable excipients are diluents, disintegrants, glidants, binders, lubricants, stabilizers, adsorbents, preservatives, release retardants or mixtures thereof.

11. The oral administration form of claim 10, wherein said pharmacologically acceptable excipients are natural starch, partially hydrolysed starch, modified starch, triethyl citrate, glycerol, potassium sorbate, lactose, glucose, sucrose, mannitol, sorbitol, cellulose and derivatives thereof, microcrystalline cellulose and derivatives thereof, calcium phosphate, calcium carbonate, calcium sulphate, polyvinylpyrrolidone, gelatin, gum tragacanth, gum arabic, polyethylene glycol, alginates, talc, magnesium stearate, silica, colloidal silica, precipitated silica, magnesium silicates, aluminium silicates, sodium lauryl sulphate, magnesium lauryl sulphate, methacrylate copolymers or mixtures thereof.

12. Unit dose of the oral administration form of claim 1, comprising 100 to 600 mg of antibiotic.

13. The unit dose of claim 12, comprising 150 to 300 mg of antibiotic.

14. The unit dose of claim 12, wherein said unit dose is a capsule.

15. Process for preparing the oral administration form of claim 1, comprising the steps of:
a) providing a solid mixture of antibiotic and excipients; and
b) applying the coating up to an amount of 6 to 20 mg/cm² on said solid mixture.

16. The process of claim 15, wherein said step b) is carried out in two sub-steps:
b-i) applying a first coating up to an amount of 4 to 8 mg/cm² on said solid mixture; and
b-ii) applying a second coating up to an amount of 2 to 12 mg/cm².

17. The process of claim 16, wherein said step b) is carried out in two sub-steps:
b-i) applying a first coating up to an amount of 5 to 7 mg/cm² on said solid mixture; and
b-ii) applying a second coating up to an amount of 2 to 12 mg/cm².

18. The process of claim 16, wherein said first coating consists of pharmaceutically acceptable excipients.

19. The process of claim 18, wherein the enteric polymer is in an amount of 50 to 80 wt. % based on the dry weight of the second coating.

20. The process of claim 16, wherein said first coating and said second coating comprise identical or different enteric polymers.

21. Method for the treatment of travellers' diarrhoea, hepatic encephalopathy, ulcerative colitis, irritable bowel syndrome (IBS), Crohn's disease, and IBD (inflammatory bowel disease) in general, as well as similar bacterial intestinal infections, comprising the step of orally administering to a subject in need thereof an effective amount of the oral administration form of claim 1.

22. The method of claim 21, wherein said treatment comprises the daily administration of the oral administration form containing 100 to 600 mg of antibiotic.

23. The method of claim 22, wherein said treatment comprises the daily administration of the oral administration form containing 150 to 300 mg of antibiotic.
